(19) United States
(12) Patent Application Publication
(10) Pub. No.: US 2014/0213971 A1
(43) Pub. Date: Jul. 31, 2014

(54) NERVE IMPINGEMENT SYSTEMS INCLUDING AN INTRAVASCULAR PROSTHESIS AND AN EXTRAVASCULAR PROSTHESIS AND ASSOCIATED SYSTEMS AND METHODS

(71) Applicants: Mark J. Dolan, Santa Rosa, CA (US); Lance Ensign, Santa Rosa, CA (US); Joseph Berglund, Santa Rosa, CA (US); Xin Weng, Santa Rosa, CA (US); Lori Garcia, Santa Rosa, CA (US); Benjamin J. Clark, Santa Rosa, CA (US)

(72) Inventors: Mark J. Dolan, Santa Rosa, CA (US); Lance Ensign, Santa Rosa, CA (US); Joseph Berglund, Santa Rosa, CA (US); Xin Weng, Santa Rosa, CA (US); Lori Garcia, Santa Rosa, CA (US); Benjamin J. Clark, Santa Rosa, CA (US)

(21) Appl. No.: 14/114,220

(22) PCT Filed: Apr. 26, 2012
(86) PCT No.: PCT/US12/35278

§ 371 Date: Mar. 11, 2014

Related U.S. Application Data
(60) Provisional application No. 61/460,768, filed on Apr. 27, 2011.

(51) Int. Cl. A61M 29/00; A61B 18/08; A61B 18/02; A61B 18/14; A61B 18/18

(52) U.S. Cl. CPC A61M 29/00; A61B 18/14; A61B 18/18; A61B 18/02; A61B 18/082
USPC 604/104; 606/191; 606/34; 606/33; 606/21; 606/27

(57) ABSTRACT

Neuromodulation assemblies (200) include an extravascular prosthesis (202) disposed around and contacting at least a portion of an exterior surface (204) of a vessel (V) and a radially expandable intravascular prosthesis (206) contacting an interior surface (208) of the vessel. The neuromodulation assemblies are configured to compress, pinch, or squeeze a target nerve within the adventitia of the vessel between the extravascular and intravascular prostheses in order to impinge and disrupt the target nerve, thereby blocking or stopping nerve signal transduction. Neuromodulation assemblies configured in accordance with the present technology may also utilize radio-frequency energy, a drug, and/or magnetic attraction to block nerve signal transduction for neuromodulation thereof.

### CROSS-REFERENCE TO RELATED APPLICATION(S)

This application claims the benefit of U.S. Provisional Application No. 61/460,768 filed Apr. 27, 2011, and incorporated herein by reference in its entirety.

### TECHNICAL FIELD

The present technology relates to systems and methods for impinging a target nerve for neuromodulation thereof.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine spillover rates in patients with essential hypertension.

Sympathetic nerves of the kidneys terminate in the blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, i.e., renal dysfunction as a progressive complication of chronic heart failure. Pharmacologic strategies to thwart the consequences of renal sympathetic stimulation include centrally-acting sympatholytic drugs, beta blockers (intended to reduce renin release), angiotensin-converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a partially schematic isometric detail view showing a common arrangement of neural fibers relative to an artery.

FIG. 2 is a partially schematic sectional view of a vessel having a neuromodulation assembly configured in accordance with an embodiment of the present technology deployed therein, wherein the neuromodulation assembly includes an extravascular prosthesis around an exterior surface of the vessel and a radially expandable intravascular prosthesis positioned within the vessel.

FIG. 2A is a cross-sectional view taken along line A-A of FIG. 2 according to an embodiment of the present technology.

FIG. 2B is a cross-sectional view taken along line A-A of FIG. 2 according to another embodiment of the present technology.

FIG. 2C is a cross-sectional view taken along line A-A of FIG. 2 according to another embodiment of the present technology.

FIG. 3 is a perspective view of the extravascular prosthesis of FIG. 2 with the extravascular prosthesis removed from the vessel for illustrative purposes only.

FIG. 3A is a cross-sectional view taken along line A-A of FIG. 3 according to an embodiment of the present technology.

FIG. 3B is a cross-sectional view taken along line A-A of FIG. 3 according to another embodiment of the present technology.

FIG. 3C is a cross-sectional view taken along line A-A of FIG. 3 according to another embodiment of the present technology.

FIG. 4 is a perspective view of an extravascular prosthesis configured in accordance with another embodiment of the present technology, wherein the extravascular prosthesis is shown around an exterior surface of a vessel.

FIG. 5 is a perspective view of an extravascular prosthesis configured in accordance with another embodiment of the present technology, wherein the extravascular prosthesis is shown around an exterior surface of a vessel.

FIG. 6 is a perspective view of an extravascular prosthesis configured in accordance with another embodiment of the present technology, wherein the extravascular prosthesis includes an electrode for radio-frequency ablation.

FIG. 6A is a cross-sectional view taken along line A-A of FIG. 6.

FIG. 7 is a perspective view of an extravascular prosthesis configured in accordance with another embodiment of the present technology, wherein the extravascular prosthesis includes holes for drug delivery.

FIG. 7A is a cross-sectional view taken along line A-A of FIG. 7, wherein the holes for drug delivery are reservoirs that extend only partially through the wall of the extravascular prosthesis.

FIG. 7B is a cross-sectional view taken along line A-A of FIG. 7 according to another embodiment of the present technology, wherein the holes for drug delivery are through holes that extend fully through the wall of the extravascular prosthesis.

FIG. 7C is a side view of an intravascular prosthesis configured in accordance with an embodiment of the present technology, wherein the intravascular prosthesis includes holes for drug delivery.

FIG. 8 is a cross-sectional view of a neuromodulation assembly configured in accordance with an embodiment of the present technology deployed within a vessel, wherein the neuromodulation assembly includes an extravascular prosthesis and an intravascular prosthesis that are magnetically attracted to each other.

### DETAILED DESCRIPTION

The present technology is generally directed to systems and methods for impinging a target nerve for neuromodulation thereof. In particular, various embodiments of the present technology are directed to nerve impingement assemblies including an extravascular prosthesis configured to be positioned around at least a portion of the circumference of a vessel and contact an exterior surface of the vessel and a radially expandable intravascular prosthesis having a generally tubular cylindrical body configured to contact an interior surface of the vessel. In operation, the intravascular prosthesis is radially positioned within the extravascular prosthesis and the nerve impingement system is configured to compress a nerve within the vessel between the extravascular and intravascular prostheses when the intravascular prosthesis is in a radially expanded configuration.

The present technology is further directed to methods of impinging nerves to induce neuromodulation. In one embodiment, for example, an extravascular prosthesis is positioned around at least a portion of the circumference of a vessel at a treatment site, and a radially expandable intravascular prosthesis is radially positioned within the extravascular prosthesis at the treatment site. The extravascular prosthesis can be deployed into contact with an exterior surface of the vessel and the intravascular prosthesis can be radially expanded into contact with an interior surface of the vessel to compress a nerve within the vessel between the extravascular and intravascular prostheses.

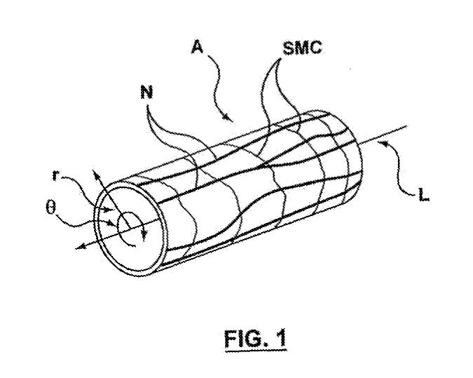

Specific details of several embodiments of the technology are described below with reference to FIGS. 1-8. Although many of the embodiments are described below with respect to devices, systems, and methods for impingement of renal nerves using extravascular and intravascular prostheses, other applications and other embodiments in addition to those described herein are within the scope of the technology. For example, although the description of the technology is in the context of treatment of blood vessels such as the coronary, carotid, and renal arteries, the technology may also be used in any other body passageways where it is deemed useful. Embodiments hereof relate to a nerve impingement assembly for neuromodulation of a targeted nerve. Embodiments of the nerve impingement assembly may be temporarily or chronically implanted within a patient and are intended to mechanically disrupt nerve conduction by applying pressure on the nerve. The biological reaction of the applied pressure may include one or more of an interruption of the nerve pathway, creation of scar tissue, tissue growth, edema formation, and other biological reactions, one or more of which may contribute to disrupting nerve conduction. There is no intention to be bound by any expressed or implied theory presented in the present disclosure. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-8.

As used herein, the terms “distal” and “proximal” define a position or direction with respect to the treating clinician or clinician's control device (e.g., a handle assembly). “Distal” or “distally” are a position distant from or in a direction away from the clinician or clinician's control device. “Proximal” and “proximally” are a position near or in a direction toward the clinician or clinician's control device.

FIG. 1 is a partially schematic isometric view of a common anatomical arrangement of neural structures relative to body lumens or vascular structures, typically arteries. Neural fibers N generally may extend longitudinally along a lengthwise or longitudinal dimension L of an artery A about a relatively small range of positions along the radial dimension r, often within the adventitia of the artery. The artery A has smooth muscle cells SMC that surround the arterial circumference and generally spiral around the angular dimension θ of the artery, also within a relatively small range of positions along the radial dimension r. The smooth muscle cells SMC of the artery A accordingly have a lengthwise or longer dimension generally extending transverse (i.e., non-parallel) to the lengthwise dimension of the blood vessel.

In various embodiments of the present technology, neural fibers are impinged or pinched to induce neuromodulation. Nerve impingement relates to compression of a nerve, and the term “pinched nerve” is often used to describe the impaired function of a nerve that is under pressure. If a nerve gets pinched, there is an interruption in conduction of the impulse down the nerve fiber. Thus, impingement of a renal nerve blocks or reduces nerve signal conduction and is expected to disrupt the sympathetic nervous system. Such modulation of renal nerve activity may be effective for treating a variety of renal and cardio-renal diseases including, but not limited to, hypertension, heart failure, renal disease, renal failure, contrast nephropathy, arrhythmia and myocardial infarction. Further, the disclosed techniques for nerve impingement may not necessarily damage the tissue or create scar tissue to block or disrupt nerve conduction.

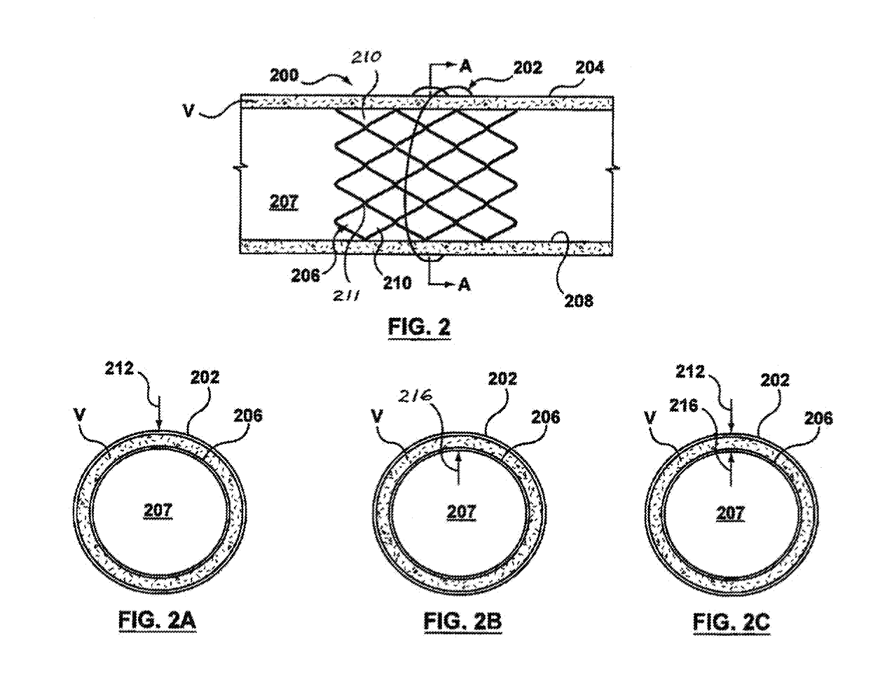

FIG. 2 is a partially schematic sectional view of a vessel V having a neuromodulation or nerve impingement assembly 200 configured in accordance with an embodiment of the present technology deployed around the vessel V. Neuromodulation assembly 200 includes an extravascular prosthesis 202 disposed around and contacting at least a portion of an exterior surface 204 of vessel V and a radially expandable intravascular prosthesis 206 contacting an interior surface 208 of the vessel V and radially positioned within the extravascular prosthesis 202. Neuromodulation assembly 200 is configured to compress, squeeze, or otherwise pinch a target nerve within the adventitia of the vessel V between the extravascular and intravascular prostheses 202, 206 in order to impinge and disrupt the target nerve, thereby blocking or stopping nerve signal transduction.

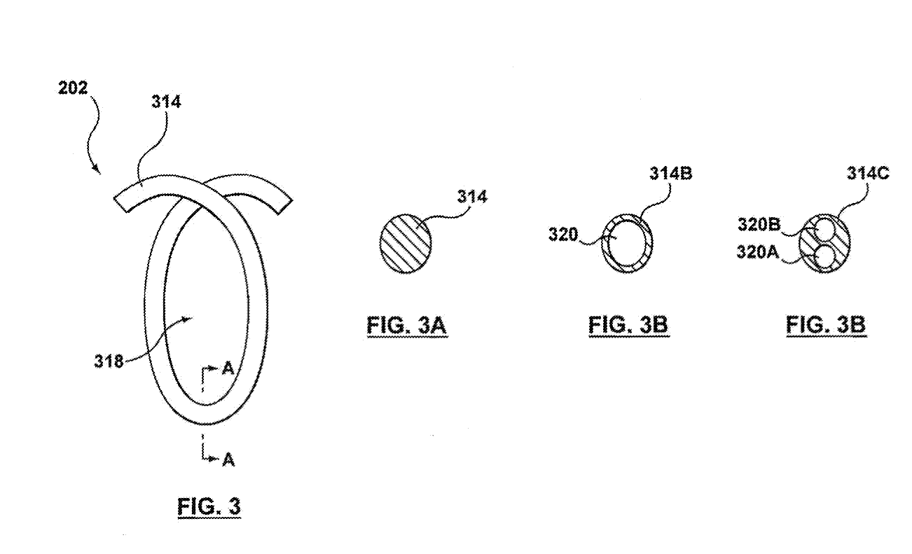

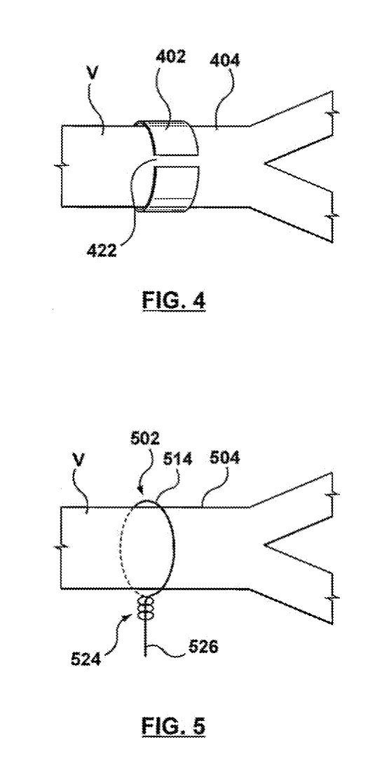

In one embodiment, the neuromodulation assembly 200 is configured to exert a compression pressure of between 40 mmHg and 400 mmHg onto the vessel V in order to impinge a nerve. Compression required for nerve impingement results from a radial pressure that may be applied by the extravascular prosthesis 202, the intravascular prosthesis 206, or both. More particularly, in one embodiment depicted in the cross-sectional view of FIG. 2A, extravascular prosthesis 202 is configured to exert a radial pressure in a radially inward direction represented by directional arrow 212. Intravascular prosthesis 206 is configured to provide resistance against the pressure exerted by extravascular prosthesis 202 onto the vessel V, and target nerve(s) within the vessel wall of the artery are thereby compressed and impinged. In addition to providing resistance against extravascular prosthesis 202, intravascular prosthesis 206 is also configured to maintain the integrity of vessel lumen 207 and may prevent collapse of the vessel V that would otherwise occur as a result of the compression exerted by extravascular prosthesis 202. When extravascular prosthesis 202 is a self-contracting coil as shown in FIG. 2 and FIG. 3 and described in more detail herein, a deployed or contracted diameter of extravascular prosthesis 202 may be predetermined to exert the required amount of inwardly-directed radial pressure in order to result in nerve impingement. More particularly, an expanded or deployed outer diameter of intravascular prosthesis 206 may be predetermined to be approximately equal to or slightly smaller or slightly larger than an inner diameter of the target vessel, i.e., a diameter of the vessel lumen. The expanded outer diameter of intravascular prosthesis 206 may be controlled via expansion of a balloon (not shown), if intravascular prosthesis 206 is balloon-expandable as described herein, or may be predetermined if intravascular prosthesis 206 is self-expanding as described herein. The contracted or deployed inner diameter of extravascular prosthesis 202 may be predetermined to be slightly less than an outer diameter of the target vessel, such that extravascular prosthesis 202 compresses the vessel V against intravascular prosthesis 206 when neuromodulation assembly 200 is deployed at a treatment site. When extravascular prosthesis 202 has a different configuration as described below with respect to FIG. 4 and FIG. 5, the extravascular prosthesis 202 may be configured to utilize alternative tightening mechanisms to exert the required amount of inwardly-directed radial pressure in order to achieve nerve impingement as described in more detail herein.

In another embodiment depicted in the cross-sectional view of FIG. 2B, intravascular prosthesis 206 is configured to exert a radial pressure onto the vessel V in a radially outward direction represented by the directional arrow 216. Extravascular prosthesis 202 is configured to provide resistance against the pressure exerted by intravascular prosthesis 206 onto the vessel V, and target nerve(s) within the vessel wall of the artery are thereby compressed and impinged. In this embodiment, the expanded diameter of intravascular prosthesis 206 may be predetermined to exert the required amount of radial pressure in order to result in nerve impingement. More particularly, an expanded or deployed outer diameter of intravascular prosthesis 206 may be predetermined to be slightly greater than the inner diameter of the target vessel, and the contracted or deployed inner diameter of extravascular prosthesis 202 may be predetermined to be approximately equal to or slightly larger or slightly smaller than the outer diameter of the target vessel. The expanded outer diameter of intravascular prosthesis 206 may be controlled via expansion of a balloon (not shown), if intravascular prosthesis 206 is balloon-expandable as described herein, or may be predetermined if intravascular prosthesis 206 is self-expanding as described herein. When deployed, radially expandable intravascular prosthesis 206 may be configured to enlarge the vessel diameter until the outer surface of vessel V comes into contact with extravascular prosthesis 202. Deployed intravascular prosthesis 206 is further configured to push the vessel V against extravascular prosthesis 202 to compress the vessel between the prostheses 202, 206, and thereby pinch the target nerve(s).

In yet another embodiment, nerve impingement may be caused by simultaneous, opposing radial pressures exerted onto the vessel V by the extravascular and intravascular prostheses 202, 206. More particularly, referring to the cross-sectional view of FIG. 2C, extravascular prosthesis 202 is configured to exert radial pressure in a radially inward direction represented by the directional arrow 212, and intravascular prosthesis 206 is configured to exert a radial pressure in a radially outward direction represented by the directional arrow 216. In this embodiment, an expanded or deployed outer diameter of intravascular prosthesis 206 may be predetermined to be slightly greater than the inner diameter of the target vessel, and the contracted or deployed inner diameter of extravascular prosthesis 202 may be predetermined to be slightly less than the outer diameter of the target vessel. When deployed, intravascular prosthesis 206 is configured to push against the interior surface of the vessel V and extravascular prosthesis 202 is configured to push against the exterior surface of the vessel V, thereby compressing the vessel V and target nerve(s) therebetween.

Extravascular prosthesis 202 and intravascular prosthesis 206 may be delivered by separate, distinct delivery systems as described in more detail herein. In one embodiment, for example, extravascular prosthesis 202 and intravascular prosthesis 206 are deployed simultaneously. In another embodiment, extravascular prosthesis 202 and intravascular prosthesis 206 may be deployed sequentially. If extravascular prosthesis 202 is configured to exert an inwardly-directed radial pressure against vessel V and thus onto intravascular prosthesis 206 as described herein with respect to FIG. 2A, it may be desirable to deploy intravascular prosthesis 206 prior to deployment of extravascular prosthesis 202 so that the radial pressure exerted by extravascular prosthesis 202 does tend to not collapse the vessel lumen. If intravascular prosthesis 206 is configured to exert an outwardly-directed radial pressure against vessel V and thus onto extravascular prosthesis 202 as described herein with respect to FIG. 2B, it may be desirable to deploy extravascular prosthesis 202 prior to deployment of intravascular prosthesis 206 such that intravascular prosthesis 206 does not tend to over-expand the vessel V.

It will be appreciated by those of ordinary skill in the art that intravascular prosthesis 206 of FIG. 2 is merely one embodiment of a radially expandable or self-expanding stent prosthesis and that various configurations of intravascular prosthesis 206 may be utilized herein. In the illustrated embodiment, for example, intravascular prosthesis 206 is a patterned, generally tubular or cylindrical expandable body that includes a plurality of cylindrical rings 210. In one embodiment, for example, cylindrical rings 210 may be formed by laser cutting or etching the entire stent body from a hollow tube or sheet in a wavelike or sinusoidal pattern, such that intravascular prosthesis 206 is a unitary structure. One of ordinary skill in the pertinent art will appreciate that intravascular prosthesis 206 can have any number of cylindrical rings 210 depending upon the desired length thereof. In another embodiment, adjacent cylindrical rings 210 may be separate wavelike or sinusoidal components formed via laser cutting, etching, or known wire forming techniques that are aligned and coupled together via at least one connection 211 to form the tubular body of intravascular prosthesis 206. Connections 211 are preferably formed by fusing the crowns together with a laser, or may alternatively be fused together via resistance welding, friction welding, soldering, by the addition of a connecting element, or by another mechanical method. Other suitable examples of stents and self-expanding and balloon-expandable stents which are suitable for use in embodiments hereof are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau, and U.S. Pat. No. 6,730,116 to Wolinsky et al., each of which is incorporated by reference herein in its entirety.

Typical materials used for intravascular prosthesis 206 are metals or alloys, examples of which include, but are not limited to, stainless steel, nickel-titanium (nitinol), cobalt-chromium, tantalum, nickel, titanium, aluminum, polymeric materials, age-hardenable nickel-cobalt-chromium-molybdenum alloy, titanium ASTM F63-83 Grade 1, niobium, platinum, gold, silver, palladium, iridium, molybdenum combinations of the above, and the like. Once implanted, the metallic stent struts can provide artificial radial support to the wall tissue. In one embodiment, for example, the intravascular and/or extravascular prostheses may be fabricated from bioabsorbable materials that will hydrolyze or corrode once placed in the body. Non-exhaustive exemplary bioabsorbable materials include, but are not limited to, magnesium, iron, zinc, magnesium-based alloys, polylactide, polyglycolide, polycaprolactone, polyurethane, co-polymers, and blends thereof.

Intravascular prosthesis 206 has an unexpanded configuration having a delivery profile sufficiently small for delivery to the treatment site within a catheter-based delivery system or other minimally invasive delivery system (not shown) and has an expanded or deployed configuration in which intravascular prosthesis 206 comes into contact with the vessel V. Embodiments of intravascular prosthesis 206 may be expanded in several ways. In one embodiment, for example, intravascular prosthesis 206 may be balloon-expandable. Intravascular prosthesis 206 may be collapsed to a contracted or compressed configuration around the balloon of a balloon dilation catheter (not shown) for delivery to a treatment site, such as the type of balloon used in an angioplasty procedure. As the balloon expands, it physically forces intravascular prosthesis 206 to radially expand such that an outside surface of intravascular prosthesis 206 comes into contact with the lumen wall. The balloon may then be collapsed leaving intravascular prosthesis 206 in the expanded or deployed configuration. Conventional balloon catheters that may be used in the present invention include any type of catheter known in the art, including over-the-wire catheters, rapid-exchange catheters, core wire catheters, and any other appropriate balloon catheters. For example, conventional balloon catheters such as those shown or described in U.S. Pat. No. 6,736,827, U.S. Pat. No. 6,554,795, U.S. Pat. No. 6,500,147, and U.S. Pat. No. 5,458,639, which are incorporated by reference herein in their entirety, may be used as the delivery system for intravascular prosthesis 206.

In another embodiment, intravascular prosthesis 206 may be self-expanding. For example, deployment of intravascular prosthesis 206 may be facilitated by utilizing thermal shape memory characteristics of a material such as nickel-titanium (nitinol). More particularly, shape memory metals are a group of metallic compositions that have the ability to return to a defined shape or size when subjected to certain thermal or stress conditions. Shape memory metals are generally capable of being deformed at a relatively low temperature and, upon exposure to a relatively higher temperature, return to the defined shape or size they held prior to the deformation. This enables the stent to be inserted into the body in a deformed, smaller state so that it assumes its “remembered” larger shape once it is exposed to a higher temperature, i.e., body temperature or heated fluid, in vivo. Thus, self-expanding intravascular prosthesis 206 can have two states of size or shape, i.e., a contracted or compressed configuration sufficient for delivery to the treatment site, and a deployed or expanded configuration having a generally cylindrical shape for contacting the vessel V.

In another embodiment in which intravascular prosthesis 206 is self-expanding, intravascular prosthesis 206 may be constructed out of a spring-type or superelastic material such as nickel-titanium (nitinol), using the stress induced martensite (SIM) properties of the material rather than the thermal shape memory properties. The catheter-based delivery system (not shown) may utilize a sheath to surround and constrain intravascular prosthesis 206 in a contracted or compressed position. Once intravascular prosthesis 206 is in position within the target vessel, the sheath may be retracted thus releasing intravascular prosthesis 206 to assume its expanded or deployed configuration.

As best seen in FIGS. 2 and 3, extravascular prosthesis 202 may comprise a coil that surrounds and/or compresses vessel V. Coiled extravascular prosthesis 202 may be formed from a wire-like component 314 shaped into a helical or corkscrew-shaped configuration that defines a vessel receiving lumen 318 through the open center of the helix. Wire-like component 314 may be solid as shown in FIG. 3A, or may be a hollow tube 314B defining a lumen 320 as shown in FIG. 3B. Although coiled extravascular prosthesis 202 is shown with a single complete winding or loop, it will be apparent to those of ordinary skill in the art that coiled extravascular prosthesis 202 may have multiple adjacent windings in either a stacked or spaced-apart form. In addition, in another embodiment hereof (not shown), the winding or loop of coiled extravascular prosthesis 202 may extend only partially around the circumference of a vessel in order to preserve vein function of an adjacent vein, as described in more detail with respect to the cuff embodiment of FIG. 4. In addition, in another embodiment hereof (not shown), the winding or loop of coiled extravascular prosthesis 202 may include loops of either uniform or varying diameter or thickness.

Wire-like component 314 may be formed of a shape-memory material that permits coiled extravascular prosthesis 202 to be substantially straightened or stretched for delivery to the treatment site and that returns the prosthesis to its original formed helical shape depicted in FIGS. 2 and 3. In order to self-form, wire-like component 314 of coiled extravascular prosthesis 202 may be made from a metallic material having a mechanical memory to return to the helical expanded configuration. Mechanical memory may be imparted to wire-like component 314 by thermal treatment to achieve a spring temper in stainless steel, for example, or to set a shape memory in a susceptible metal alloy, such as nitinol. In an alternate embodiment, a mechanical memory (to return to the helical expanded configuration) may be imparted to a polymer that forms wire-like component 314, such as any of the polymers disclosed in U.S. Pat. Appl. Pub. No. 2004/0111111 to Lin, which is incorporated herein by reference in its entirety.

In another embodiment shown in FIG. 3C, wire-like component 314 of coiled extravascular prosthesis 202 may be a tubular component 314C defining a first lumen 320A and a second lumen 320B. Dual lumens 320A, 320B may be utilized for circulating a heating fluid for deploying extravascular prosthesis 202 into its coiled, contracted configuration to surround and/or compress around the vessel V (FIG. 2). Stated another way, the deployed or contracted configuration of extravascular prosthesis 202 may be achieved by utilizing temperature-dependent characteristics of a material. More particularly, some shape memory metals have the ability to return to a defined shape or size when subjected to certain thermal or stress conditions. Shape memory metals are generally capable of being deformed at a relatively low temperature and, upon exposure to a relatively higher temperature, return to the defined shape or size they held prior to the deformation. Extravascular prosthesis 202 may be deformed into the straightened configuration when delivered to the treatment site. Upon reaching a treatment site within a body lumen and being loosely positioned around the exterior circumference of the vessel, heated fluid may be circulated through extravascular prosthesis 202 via dual lumens 320A, 320B such that extravascular prosthesis 202 is allowed to assume its “remembered” expanded configuration in vivo. Therefore, coiled extravascular prosthesis 202 may be caused to tighten or compress around the vessel via temperature control.

In order to chronically implant a coiled extravascular prosthesis that is deployed via temperature control, the prosthesis may be detachably connected to a fluid supply shaft (not shown) and a fluid return shaft (not shown). The fluid supply shaft defines a lumen that is in fluid communication with one of dual lumens 320A, 320B of tubular component 314C, and the fluid return shaft defines a lumen that is in fluid communication with the other of dual lumens 320A, 320B. In one embodiment, sleeves (not shown) may surround or cover the connections between coiled extravascular prosthesis 202 and the fluid supply and fluid return shafts. The sleeves may be formed from a material having a higher melting temperature than a temperature of the heated fluid. After deployment of coiled extravascular prosthesis 202, a heater (not shown), such as a dual wire heater, may be distally advanced through the lumen of the fluid supply shaft to the connection between coiled extravascular prosthesis 202 and the fluid supply shaft. An electrical current may then be delivered to the heater to melt the sleeve, thus separating or disconnecting the fluid supply shaft from extravascular prosthesis 202. This process is then repeated for severing the connection between the fluid return shaft and extravascular prosthesis 202. In addition to severing the connections between the fluid supply and return shafts and the extravascular prosthesis, the electrical current may also result in resistive heating that may degrade the tissue of the vessel, thereby making it more susceptible to compression.

Referring back to FIG. 2, coiled extravascular prosthesis 202 may be delivered by any suitable delivery system. In one embodiment, for example, coiled extravascular prosthesis 202 is intravascularly delivered by a catheter device (not shown) having a side port for delivering wire-like component 314 through a perforation in the vessel wall to an extravascular position. The perforation in the vessel wall may be formed via the catheter device, or via a separate intravascular device. In one embodiment, for example, a suitable delivery catheter that may be modified for use herein is the PIONEER catheter produced by Medtronic, Inc. of Minneapolis, Minn. To deliver a self-expanding coiled extravascular prosthesis, the coiled extravascular prosthesis may be substantially straightened into a delivery configuration and distally advanced out of the side port of the catheter and through a perforation in the vessel. As the substantially straightened coiled extravascular prosthesis passes through the vessel wall, once clear of the delivery system support, its pre-shaped form coils around the outer surface of the vessel until the distal end thereof exits the catheter device and the coiled extravascular prosthesis at least partially encircles the exterior of the vessel. The substantially straightened coiled extravascular prosthesis may be distally advanced through the side port of the catheter via a pusher tube or rod that extends the full length of the catheter, with the proximal end thereof extending outside of the patient. In another embodiment, coiled extravascular prosthesis 202 may be delivered in an extravascular approach via a laparoscopic tool which is capable of gaining access to the exterior circumference of a target vessel.

FIG. 4 is a perspective view of an extravascular prosthesis 402 configured in accordance with another embodiment of the present technology deployed around an exterior surface 404 of vessel V. Although not shown in this view, extravascular prosthesis 402 is intended to be utilized with intravascular prosthesis 206 in order to compress a portion of vessel V therebetween. In this embodiment, extravascular prosthesis 402 comprises a C-clamp or cuff that does not encircle the full circumference of vessel V. A gap or space 422 exists between opposing ends of extravascular prosthesis 402. In some embodiments, extravascular prosthesis 402 may encircle between 60-95% of the circumference of vessel V. In one particular embodiment, for example, extravascular prosthesis 402 encircles approximately 75% of the circumference of vessel V.

Extravascular prosthesis 402 may be utilized to preserve vein function. More particularly, extravascular prosthesis 402 may be positioned around vessel V (e.g. an artery) such that gap 422 of extravascular prosthesis 402 (rather than the cuff structure) is located against an adjacent vein. Since the cuff structure does not contact or engage the adjacent vein, vein function is not expected to be altered by the presence of extravascular prosthesis 402. Extravascular prosthesis 402 is configured to be extravascularly delivered and positioned around vessel V. To exert the radial pressure on the vessel required for neuromodulation as described above with respect to FIG. 2A and FIG. 2C, extravascular prosthesis 402 may be squeezed or compressed by a clinician to tighten extravascular prosthesis 402 around the vessel V in order exert the required amount of radial pressure to result in nerve impingement. In one embodiment, for example, extravascular prosthesis 402 may be delivered using an extravascular approach via a laparoscopic tool that is capable of gaining access to an exterior circumference of target vessel V. In another embodiment, extravascular prosthesis 402 may be delivered to the treatment site in the shape of a hook having a bend of approximately 180° and then crimped into the C-shape with a surgical tool similar to a laparoscopic tenaculum.

Extravascular prosthesis 402 may be formed from a shape-memory material such as those listed herein that permits extravascular prosthesis 402 to be substantially straightened or stretched for delivery to the treatment site, and that returns extravascular prosthesis 402 to its original expanded C-shape depicted in FIG. 4. When returning to its original expanded C-shape, extravascular prosthesis 402 is configured to at least partially encircle the exterior surface of the vessel V.

FIG. 5 is a perspective view of an extravascular prosthesis 502 configured in accordance with another embodiment of the present technology deployed around an exterior surface 504 of vessel V. Although not shown in this view, extravascular prosthesis 502 is intended to be utilized with an intravascular prosthesis, such as intravascular prosthesis 206, in order to compress a portion of vessel V therebetween. Extravascular prosthesis 502 may be formed from an elongated suture-like component 514 and operates in a noose-like fashion. More particularly, a first or distal end of suture-like component 514 can include a preformed loop or hook 524 thereon that is configured to catch or receive suture-like component 514 therethrough. The distal end of suture-like component 514 may be wrapped around an exterior circumference of a vessel V and hook 524 may be manipulated to catch suture-like component 514 therein, such that suture-like component 514 encircles or surrounds the vessel V. A second or proximal end (not shown) of suture-like component 514 extends proximally outside of a patient to be manipulated by an operator. To exert the radial pressure on the vessel V required for neuromodulation as described above with respect to FIG. 2A and FIG. 2C, extravascular prosthesis 502 may be tightened by a clinician to constrict extravascular prosthesis 502 around the vessel in order exert sufficient radial pressure to result in nerve impingement. More particularly, suture-like component 514 may be slidingly disposed through hook 524 such that once the circular portion of suture-like component 514 formed by hook 524 encircles the vessel V, the operator may apply a pulling force to the proximal end of suture-like component 514 in a proximal direction in order to tighten extravascular prosthesis 502.

If extravascular prosthesis 502 is intended to be chronically implanted, suture-like component 514 may be tied off proximal to hook 524 and cut as shown in FIG. 5 by a severed end 526. Extravascular prosthesis 502 is extravascularly delivered and positioned around vessel V. In one embodiment, for example, extravascular prosthesis 502 may be delivered using an extravascular approach via a laparoscopic tool similar to a laparoscopic tenaculum that is capable of gaining access to the exterior circumference of a target vessel. The laparoscopic tool can include an embedded or preloaded suture-like component therein. Once suture-like component 514 is wrapped around the target vessel, the ends of suture-like component 514 may be captured using cuffs that are built into the laparoscopic tool similar to the CLOSER S suture closure device produced by Perclose/Abbott Laboratories of Abbott Park, Ill. The ends of suture-like component 514 may then be threaded outside the body for easy access by the physician, after which a knot is tied. The knot can then be slid in a distal direction until it abuts against the vessel V to tighten extravascular prosthesis 502 around the vessel V as desired.

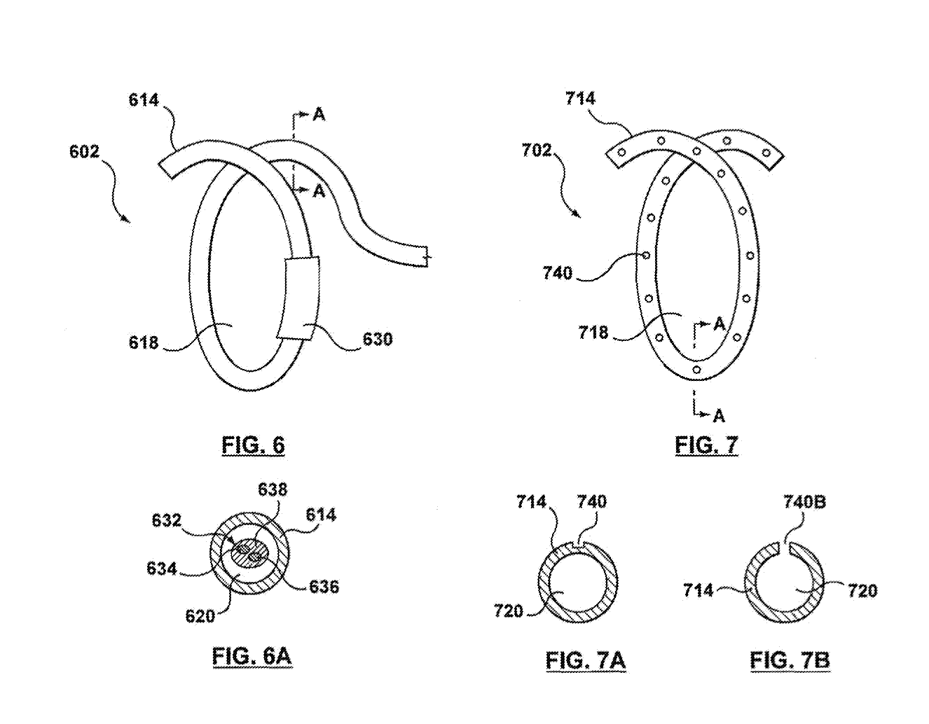

In addition to vessel wall pressure generated between the extravascular and intravascular prostheses, neuromodulation assemblies configured in accordance with the present technology may also utilize radio-frequency energy, a thermal fluid, a drug, and/or magnetic attraction to block nerve signal transduction for neuromodulation thereof. FIG. 6, for example, illustrates an embodiment of the present technology in which ablative energy is utilized in addition to pressure between the extravascular and intravascular prostheses for neuromodulation of a targeted nerve. More particularly, FIG. 6 illustrates a coiled extravascular prosthesis 602 configured to surround and/or compress a vessel (not shown) in conjunction with an intravascular prosthesis (not shown) as described above with respect to FIG. 2. Coiled extravascular prosthesis 602 may be formed from a wire-like component 614 shaped into a helical or corkscrew-like configuration that defines a vessel receiving lumen 618 through the open center of the helix. Coiled extravascular prosthesis 602 can also include at least one electrode 630 for selectively delivering ablation energy from an external generator or power supply (not shown) to a vessel. In another embodiment (not shown), wire-like component 614 itself may be formed from a suitable material in order to act as the electrode for delivering ablation energy from the generator. In one embodiment, for example, the generator may be a multi-channel radio frequency generator such as the GENIUS generator produced by Medtronic Ablation Frontiers of Carlsbad, Calif. The ablation energy delivered through electrode 630 is expected to cause ablation of at least a portion of the vessel V, thereby blocking nerve signal transduction to assist in neuromodulation of targeted nerves.

In the illustrated embodiment, electrode 630 is a band electrode, which has lower power requirements for ablation as compared to disc or flat electrodes. Disc or flat electrodes, however, are also suitable for use herein. In another embodiment, electrodes having a spiral or coil shape may be utilized. Electrode 630 may be formed from any suitable metallic material including gold, platinum or a combination of platinum and iridium. In the embodiment depicted in FIG. 6, coiled extravascular prosthesis 602 includes a single electrode, but it will be apparent to one of ordinary skill in the art that a plurality of electrodes may be utilized. In addition, if a plurality of electrodes are utilized, it is not required that the electrodes be equally spaced apart but rather the distance between the electrodes may vary depending on the particular application. For example, the desired ablation pattern, i.e., a full circumferential ablation pattern, a partial circumferential ablation pattern, or a non-continuous circumferential ablation pattern, may dictate the desired spacing of the electrodes, i.e., the distance between the electrodes as well as whether the electrodes are equally spaced apart or variably spaced apart. It will be understood by one of ordinary skill in the art that the length of electrode 630 may vary according to its intended application.

Each electrode of coiled extravascular prosthesis 602 is electrically connected to the generator by a conductor or wire 632 that extends through lumen 620 of hollow wire-like component 614, as shown in FIG. 6A. Since the embodiment of FIG. 6 includes only one electrode, only one corresponding bifilar wire 632 is required to electrically connect electrode 630 to a generator (not shown). In embodiments including multiple electrodes, additional wires may be carried by the extravascular prosthesis 602 and electrically coupled to the generator. Each electrode may be welded or otherwise electrically coupled to the distal end of its respective wire 632, and each wire 632 can extend proximally out of the patient such that a proximal end thereof is coupled to the generator. In the embodiment shown in FIG. 6A, each wire 632 is a bifilar wire that includes a first conductor 634, a second conductor 636, and insulation 638 surrounding each conductor to electrically isolate them from each other. In one particular embodiment, first conductor 634 may be a copper conductor, second conductor 636 may be a copper/nickel conductor, and insulation 638 may be polyimide insulation. In other embodiments, however, the wire 632 may have a different configuration and/or be composed of different materials.

When coupled to an electrode (e.g., electrode 630), the two conductors of bifilar wire 632 function to provide power to its respective electrode and act as a T-type thermocouple for the purposes of measuring the temperature of the electrode 630. Temperature measurement provides feedback to the generator such that the power delivered to each electrode 630 can be automatically adjusted by the generator to achieve a target temperature, and also provides an indication of the quality of the contact between the electrode and the adjacent tissue. In one embodiment, during the ablation procedure the generator may display the power each electrode 630 is receiving and the temperature achieved such that the user may assess each electrode's tissue contact. In another embodiment, wire 632 may be a single conductor wire rather than a bifilar wire described above. Each single conductor wire provides power to its respective electrode, but does not measure the temperature of the electrode.

After the ablation energy is delivered, electrode(s) 630 may be configured to detach or disconnect from coiled extravascular prosthesis 602 to allow for chronic implantation of the prosthesis. In one embodiment, for example, electrode(s) 630 may be connected to coiled extravascular prosthesis 602 via a detachable connection such as a solder joint having a melting point approximately equal to the temperature of the ablation energy. Once the ablation energy is delivered, the solder joint heats to a temperature of the ablation energy and since this temperature is the solder melting point, the joint breaks. Once the solder joint breaks, electrode(s) 630 disconnect from extravascular prosthesis 602 so that they may be pulled out and removed from the patient, leaving coiled extravascular prosthesis 602 in place.

In another embodiment, a thermal agent such as a fluid or gas may be utilized in addition to the vessel wall pressure generated between the extravascular and intravascular prostheses for neuromodulation of a targeted nerve. Referring back to FIG. 3 and FIG. 3C, for example, dual lumens 320A, 320B of wire-like component 314 may be utilized for continuously circulating a heating or cooling agent that assists in neuromodulation of targeted nerves. As described in U.S. Pat. No. 7,617,005 to Demarais et al. and U.S. Patent Appl. Pub. No. 2007/0129720 to Demarais et al., both of which are currently commonly owned by the assignee of the present technology and herein incorporated by reference in their entirety, heating or cooling causes thermal stress that may affect or alter the neural structures, thereby causing thermal neuromodulation. In one embodiment, the cooling agent may have freezing or cryotherapy temperatures to thermally damage or ablate target tissue of an artery to achieve neuromodulation of the target neural fibers. In addition or alternatively, the heating or cooling agent also may degrade the tissue of the vessel thereby making it more susceptible to compression.

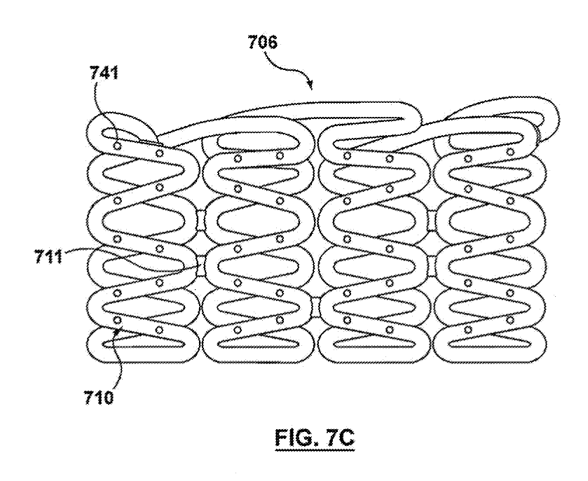

FIG. 7 illustrates an embodiment in which drug delivery is utilized in addition to vessel wall pressure generated between the extravascular and intravascular prostheses for neuromodulation of a targeted nerve. More particularly, FIG. 7 illustrates a coiled extravascular prosthesis 702 configured to surround and/or compress vessel V in conjunction with an intravascular prosthesis (not shown) as described above with respect to FIG. 2. Coiled extravascular prosthesis 702 is formed from a wire-like component 714 shaped into a helical or corkscrew-like configuration that defines a vessel receiving lumen 718 through the open center of the helix. Coiled extravascular prosthesis 702 can include a plurality of drug delivery holes 740 for delivering a therapeutic substance or drug to a vessel, which enhances neuromodulation of targeted nerve(s). In one embodiment, for example, drug delivery holes 740 may be located on an interior surface of the helix or coil such that the therapeutic substance is directionally delivered to the exterior surface of the vessel. In one embodiment, the drug that enhances neuromodulation of targeted nerve(s) is a neurotoxin drug that is specific to block signal transduction to the targeted nerve(s) such as, but not limited to, botulinun neurotoxin, batrachotoxin, tetrodotoxin, and phoneutria nigriventer toxin-3 (PhTx3). In another embodiment, the drug that enhances neuromodulation of targeted nerve(s) is a softening drug that makes the vessel more susceptible to compression such as, but not limited to, collagenase, elastase, cathepsin G, pepsin, and metalloproteinases. The softening drug is expected to enhance the efficiency of impingement of the nerve via pressure between the extravascular and intravascular prostheses.

In an embodiment shown in FIG. 7A, drug delivery holes 740 may be reservoirs formed within an outer surface of wire-like component 714 for holding a therapeutic substance or drug therein. Holes or reservoirs 740 can have a depth that extends from an exterior surface of wire-like component 714 to approximately midway through the wall of wire-like component 714 and the therapeutic substance or drug is located therein.

In another embodiment shown in FIG. 7B, wire-like component 714 may include a central lumen or fluid passageway 720 for holding a therapeutic substance or drug therein. Drug delivery holes 740B, for example, may be passageways or thru-holes formed through wire-like component 714 that allow for elution of the therapeutic substance or drug stored within lumen 720. Passageways or thru-holes 740B can have a depth that extends from an interior surface of wire-like component 714 to an exterior surface of wire-like component 714 so that the therapeutic substance or drug located in central lumen 720 may be delivered to a vessel. In one embodiment, the elutable therapeutic substance or drug may be pre-loaded into central lumen 720 prior to implantation into the body, with both ends of wire-like component 714 being closed once the drug is loaded. The term “pre-loaded” as used herein means that, prior to delivery into the body vessel, a therapeutic substance or drug may be filled, injected, or otherwise provided within drug delivery reservoirs 740 or central lumen 720 of wire-like component 714, after which ends of wire-like component 714 are sealed or plugged.

In addition to or as an alternative to drug delivery via extravascular prosthesis 702, the intravascular prosthesis of the neuromodulation assembly may be used for delivering any suitable therapeutic substance to the walls and/or interior of a body vessel to assist in or enhance neuromodulation of a targeted nerve. FIG. 7C, for example, illustrates an intravascular prosthesis 706 configured to be radially deployed within a vessel in conjunction with any one of the extravascular prostheses described herein. In the illustrated embodiment, intravascular prosthesis 706 is a patterned generally tubular or cylindrical expandable body that includes a plurality of cylindrical rings 710 coupled together at connections 711. Intravascular prosthesis 706 can also include a plurality of drug delivery holes 741 for delivering a therapeutic substance or drug to a vessel, which is expected to enhance neuromodulation of targeted nerve(s). In one embodiment, for example, drug delivery holes 741 may be located on an exterior surface of the cylindrical body such that the therapeutic substance is directionally delivered to the interior surface of the vessel. It will be appreciated by one of ordinary skill in the art that the depiction of intravascular prosthesis 706 in FIG. 7C is merely by way of example, and that any of the stents described above could be modified to include drug delivery holes 741 to be suitable for use in accordance with embodiments hereof.

As described above with respect to drug delivery holes 740 in extravascular prosthesis 702, drug delivery holes 741 of intravascular prosthesis 706 may be reservoirs as shown in FIG. 7A, or may be thru-holes in fluid communication with a central lumen as shown in FIG. 7B. Similarly, as described above with respect to extravascular prosthesis 702, the delivered therapeutic substance may be a neurotoxin drug that is specific to block signal transduction to the targeted nerve(s) or may be a softening drug that makes the vessel more susceptible to compression.

In various embodiments of the present technology, the elutable therapeutic substance or drug contained in the extravascular and/or intravascular prostheses may comprise a biologically or pharmacologically active substance. In one embodiment, for example, the elutable therapeutic substance or drug may be in crystalline form. In another embodiment, the biologically or pharmacologically active substance may be suspended in a polymer matrix or carrier to prevent premature elution of the active therapeutic substance from the drug delivery holes until after the extravascular prosthesis and/or the intravascular prosthesis have been implanted at the treatment site. Methods of making a polymer carrier or matrix for biologically or pharmacologically active ingredients are well known in the art. For example, biologically or pharmacologically active substances and carriers for these substances are listed in U.S. Pat. No. 6,364,856, U.S. Pat. No. 6,358,556, and U.S. Pat. No. 6,258,121, each of which is incorporated by reference herein in its entirety. These patent references disclose active substances, as well as polymer materials impregnated with the active substances for use as coatings on the outside of medical devices to provide controlled delivery of the active substances. These same polymer materials impregnated with active substances may be used within drug delivery reservoirs or a central lumen of an extravascular and/or intravascular prosthesis in accordance with embodiments hereof. In one embodiment, for example, the polymer matrix or carrier may be biodegradable or bioresorbable such that it is absorbed in the body. Polylactic acid (PLA), polyglycolic acid, polyethylene oxide (PEO), and polycaprolactone are examples of biodegradable polymeric carriers.

In addition, a readily dissolvable coating (not shown) may be utilized in embodiments of the present technology in order to prevent premature elution of the active therapeutic substance from drug delivery reservoirs or a central lumen of an extravascular and/or intravascular prosthesis until the prosthesis has been deployed at the treatment site. The coating, for example, may cover or close up the drug delivery holes, may cover the outside surface of the prosthesis, or both. The coating may be a dextran type or any other appropriate coating that would dissolve very quickly, yet protect the therapeutic substance or drug as it is being delivered to the treatment site. For example, coating materials that may be sufficient to provide the desired short duration protection, such as polysaccharides including mannitol, sorbitol, sucrose, xylitol, anionic hydrated polysaccharides such as gellan, curdlan, extracellular anionic 1,3-linked glycan (XM-6), xanthan, are listed in U.S. Pat. No. 6,391,033, which is incorporated by reference herein in its entirety. These materials may dissolve in approximately ten to fifteen minutes in order to allow for proper prosthesis placement at the target site.

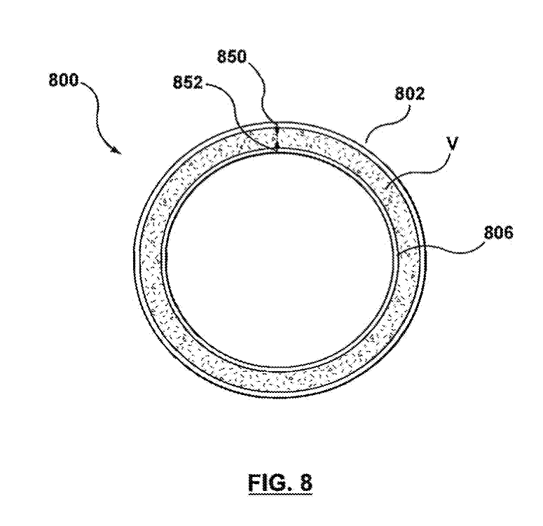

FIG. 8 is a cross-sectional view of a neuromodulation assembly 800 configured in accordance with an embodiment of the present technology deployed within a vessel V. In this embodiment, magnetism assists in compressing a targeted nerve between the extravascular and intravascular prostheses for neuromodulation thereof. More particularly, neuromodulation assembly 800 includes an extravascular prosthesis 802 and an intravascular prosthesis 806 that are magnetically attracted to each other. Magnetic force or attraction between the prostheses 802, 806 is expected to provide compression and pinching of the targeted nerve.

Extravascular and intravascular prostheses 802, 806 may each be formed of or have incorporated therein or thereon a material capable of producing a magnetic field that acts to maintain the components in a desired positional relationship. For example, the material used to form one or both extravascular and intravascular prostheses 802, 806 may be magnetic, ferromagnetic or electromagnetic. Suitable materials that may be used to form one of extravascular and intravascular prostheses 802, 806 include neodymium-iron-boron, samarium-cobalt, and aluminum-nickel-cobalt. In other embodiments, other suitable materials may be used. The strength of the magnetic field, i.e., the magnetic attractive force, exerted depends on various factors including the materials used, the size of the magnet(s), and the number of magnets. In one embodiment, one or both extravascular and intravascular prostheses 802, 806 may be coated with a magnetic coating formed from suitable ferromagnetic metals and alloys, such as cobalt, nickel, iron, or other suitable compositions having magnetic or magnetizable properties. For example, the magnetic coating may be one of the coating compositions described in U.S. Pat. No. 6,790,378, U.S. Pat. No. 7,001,645 or U.S. Pat. No. 6,673,104, the disclosures of which are incorporated by reference herein in their entirety. The magnetic coating may be applied over all or a portion of an exterior surface of one or both extravascular and intravascular prostheses 802, 806. Suitable approaches for applying the coating include various deposition methods, including, for example, sputtering, vapor deposition, metal plasma deposition, ion beam deposition, and other similar approaches.

### EXAMPLES

1. A nerve impingement system, the system comprising:
an extravascular prosthesis configured to be positioned around at least a portion of a circumference of a vessel to contact an exterior surface of the vessel; and
a radially expandable intravascular prosthesis having a generally tubular body configured to contact an interior surface of the vessel, wherein the intravascular prosthesis is radially positionable within the extravascular prosthesis in vivo such that a portion of the vessel is sandwiched thereby, and
wherein, in a deployed configuration in vivo, the nerve impingement system is configured to compress a nerve within the portion of the vessel sandwiched between the extravascular and intravascular prostheses.

2. The system of example 1 wherein an inner diameter of the extravascular prosthesis is less than an outer diameter of the vessel such that the extravascular prosthesis is configured to exert an inward radial pressure onto the intravascular prosthesis in order to compress the nerve within the vessel between the extravascular and intravascular prostheses.

3. The system of example 1 wherein an outer diameter of the intravascular prosthesis is greater than an inner diameter of the vessel such that the intravascular prosthesis is configured to exert an outward radial pressure onto the extravascular prosthesis in order to compress the nerve within the vessel between the extravascular and intravascular prostheses.

4. The system of example 1 wherein the extravascular prosthesis comprises a coil having at least one winding that encircles the circumference of the vessel.

5. The system of example 1 wherein the extravascular prosthesis comprises a cuff that encircles a portion of the circumference of the vessel.

6. The system of example 1 wherein the extravascular prosthesis includes at least one electrode thereon.

7. The system of example 1 wherein at least one of the extravascular prosthesis and the intravascular prosthesis includes a reservoir formed on an exterior surface thereof, and wherein the reservoir is configured to be filled with a therapeutic substance.

8. The system of example 1 wherein the intravascular prosthesis and the extravascular prosthesis are magnetically attracted to each other.

9. A method of impinging a nerve to achieve neuromodulation thereof, the method comprising:
positioning an extravascular prosthesis around at least a portion of a circumference of a vessel at a treatment site;
positioning a radially expandable intravascular prosthesis such that the intravascular prosthesis is radially disposed within the extravascular prosthesis at the treatment site, wherein the intravascular prosthesis has a generally tubular cylindrical body;
deploying the extravascular prosthesis into contact with an exterior surface of the vessel; and
radially expanding the intravascular prosthesis into contact with an interior surface of the vessel,
wherein the nerve is sandwiched and compressed between the extravascular and intravascular prostheses such that compression of the nerve causes neuromodulation thereof.

10. The method of example 9 wherein deploying the extravascular prosthesis is performed prior to radially expanding the intravascular prosthesis or after radially expanding the intravascular prosthesis.

11. The method of example 9 wherein positioning the extravascular prosthesis includes extravascularly delivering the extravascular prosthesis to the treatment site and placing the extravascular prosthesis around the exterior surface of the vessel at the treatment site.

12. The method of example 9 wherein positioning the extravascular prosthesis includes intravascularly delivering the extravascular prosthesis to the treatment site, advancing the extravascular prosthesis through the vessel, and placing the extravascular prosthesis around the exterior surface of the vessel at the treatment site.

13. The method of example 9 wherein positioning the intravascular prosthesis includes intravascularly delivering the intravascular prosthesis to the treatment site.

14. The method of example 9, further comprising utilizing the extravascular prosthesis to deliver radio-frequency energy to the vessel.

15. The method of example 9, further comprising utilizing the extravascular prosthesis to provide cryogenic therapy to the vessel.

16. The method of example 9, further comprising utilizing the extravascular prosthesis to provide heat therapy to the vessel.

17. The method of example 9, further comprising utilizing at least one of the extravascular prosthesis and the intravascular prosthesis to provide drug therapy to the vessel, wherein the drug therapy is a neurotoxin that blocks signal transduction of the nerve.

18. The method of example 9, further comprising utilizing at least one of the extravascular prosthesis and the intravascular prosthesis to provide drug therapy to the vessel, wherein the drug therapy acts upon the vessel to enhance the efficiency of compressing the nerve between the extravascular and intravascular prostheses.

19. The method of example 9 wherein the intravascular prosthesis and the extravascular prosthesis are magnetically attracted to each other.

20. The method of example 9 wherein deploying the extravascular prosthesis into contact with the exterior surface of the vessel includes expanding the extravascular prosthesis to an expanded diameter that is slightly smaller than an outer diameter of the vessel.

21. The method of example 9 wherein deploying the extravascular prosthesis into contact with the exterior surface of the vessel includes tightening the extravascular prosthesis to compress the vessel.

22. The method of example 9 wherein radially expanding the intravascular prosthesis includes expanding the intravascular prosthesis to an expanded diameter that is slightly larger than an inner diameter of the vessel.

### CONCLUSION

While various embodiments according to the present technology have been described above, it should be understood that they have been presented by way of illustration and example only, and not limitation. It will be apparent to persons skilled in the relevant art that various changes in form and detail can be made therein without departing from the spirit and scope of the disclosure. For example, one or more of the coils described herein could be made from an expandable material that increases in wire/tube diameter over time. More specifically, such coil(s) would have one diameter upon placement and a second, larger diameter at a later period of time (e.g., several minutes, several months, etc.). One particular example of such a material is iron. When iron oxidizes, the iron oxide doubles in volume. Other suitable materials include polymers that act like sponges and expand when they hydrolyze. Accordingly, it will be appreciated that the breadth and scope of the present technology should not be limited by any of the above-described embodiments. It will also be understood that each feature of each embodiment discussed herein, and of each reference cited herein, can be used in combination with the features of any other embodiment. All patents and publications discussed herein are incorporated by reference herein in their entirety.

Where the context permits, singular or plural terms may also include the plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

I/We claim:

1. A nerve impingement system, the system comprising:
an extravascular prosthesis configured to be positioned around at least a portion of a circumference of a vessel to contact an exterior surface of the vessel; and
a radially expandable intravascular prosthesis having a generally tubular body configured to contact an interior surface of the vessel, wherein the intravascular prosthesis is radially positionable within the extravascular prosthesis in vivo such that a portion of the vessel is sandwiched thereby, and
wherein, in a deployed configuration in vivo, the nerve impingement system is configured to compress a nerve within the portion of the vessel sandwiched between the extravascular and intravascular prostheses.

2. The system of claim 1 wherein an inner diameter of the extravascular prosthesis is less than an outer diameter of the vessel such that the extravascular prosthesis is configured to exert an inward radial pressure onto the intravascular prosthesis in order to compress the nerve within the vessel between the extravascular and intravascular prostheses.

3. The system of claim 1 wherein an outer diameter of the intravascular prosthesis is greater than an inner diameter of the vessel such that the intravascular prosthesis is configured to exert an outward radial pressure onto the extravascular prosthesis in order to compress the nerve within the vessel between the extravascular and intravascular prostheses.

4. The system of claim 1 wherein the extravascular prosthesis comprises a coil having at least one winding that encircles the circumference of the vessel.

5. The system of claim 1 wherein the extravascular prosthesis comprises a cuff that encircles a portion of the circumference of the vessel.

6. The system of claim 1 wherein the extravascular prosthesis includes at least one electrode thereon.

7. The system of claim 1 wherein at least one of the extravascular prosthesis and the intravascular prosthesis includes a reservoir formed on an exterior surface thereof, and wherein the reservoir is configured to be filled with a therapeutic substance.

8. The system of claim 1 wherein the intravascular prosthesis and the extravascular prosthesis are magnetically attracted to each other.

9. A method of impinging a nerve to achieve neuromodulation thereof, the method comprising:
positioning an extravascular prosthesis around at least a portion of a circumference of a vessel at a treatment site;
positioning a radially expandable intravascular prosthesis such that the intravascular prosthesis is radially disposed within the extravascular prosthesis at the treatment site, wherein the intravascular prosthesis has a generally tubular cylindrical body;
deploying the extravascular prosthesis into contact with an exterior surface of the vessel; and
radially expanding the intravascular prosthesis into contact with an interior surface of the vessel,
wherein the nerve is sandwiched and compressed between the extravascular and intravascular prostheses such that compression of the nerve causes neuromodulation thereof.

10. The method of claim 9 wherein deploying the extravascular prosthesis is performed prior to radially expanding the intravascular prosthesis or after radially expanding the intravascular prosthesis.

11. The method of claim 9 wherein positioning the extravascular prosthesis includes extravascularly delivering the extravascular prosthesis to the treatment site and placing the extravascular prosthesis around the exterior surface of the vessel at the treatment site.

12. The method of claim 9 wherein positioning the extravascular prosthesis includes intravascularly delivering the extravascular prosthesis to the treatment site, advancing the extravascular prosthesis through the vessel, and placing the extravascular prosthesis around the exterior surface of the vessel at the treatment site.

13. The method of claim 9 wherein positioning the intravascular prosthesis includes intravascularly delivering the intravascular prosthesis to the treatment site.

14. The method of claim 9, further comprising utilizing the extravascular prosthesis to deliver radio-frequency energy to the vessel.

15. The method of claim 9, further comprising utilizing the extravascular prosthesis to provide cryogenic therapy to the vessel.

16. The method of claim 9, further comprising utilizing the extravascular prosthesis to provide heat therapy to the vessel.

17. The method of claim 9, further comprising utilizing at least one of the extravascular prosthesis and the intravascular prosthesis to provide drug therapy to the vessel, wherein the drug therapy is a neurotoxin that blocks signal transduction of the nerve.

18. The method of claim 9, further comprising utilizing at least one of the extravascular prosthesis and the intravascular prosthesis to provide drug therapy to the vessel, wherein the drug therapy acts upon the vessel to enhance the efficiency of compressing the nerve between the extravascular and intravascular prostheses.

19. The method of claim 9 wherein the intravascular prosthesis and the extravascular prosthesis are magnetically attracted to each other.

20. The method of claim 9 wherein deploying the extravascular prosthesis into contact with the exterior surface of the vessel includes expanding the extravascular prosthesis to an expanded diameter that is slightly smaller than an outer diameter of the vessel.

21. The method of claim 9 wherein deploying the extravascular prosthesis into contact with the exterior surface of the vessel includes tightening the extravascular prosthesis to compress the vessel.

22. The method of claim 9 wherein radially expanding the intravascular prosthesis includes expanding the intravascular prosthesis to an expanded diameter that is slightly larger than an inner diameter of the vessel.
